(19) United States
(12) Patent Application Publication
(10) Pub. No.: US 2015/0104834 A1
(43) Pub. Date: Apr. 16, 2015

(54) Culture Medium and Methods for Producing Alginate From Stable Mucoid Strains of Pseudomonas Aeruginosa

(71) Applicant: Progenesis Technologies, LLC, Huntington, WV (US)

(72) Inventors: Hongwei David Yu, Huntington, WV (US); Richard M. Niles, Huntington, WV (US); Xin Wang, Hangzhou (CN); Kristy Dawn Dillon, Huntington, WV (US)

(21) Appl. No.: 14/515,558

(22) Filed: Oct. 16, 2014

Related U.S. Application Data
(63) Continuation of application No. 13/348,897, filed on Jan. 12, 2012, now abandoned.
(60) Provisional application No. 61/432,762, filed on Jan. 14, 2011.

(51) Int. Cl. C12P 19/04; C12N 1/20

(52) U.S. Cl. CPC C12P 19/04; C12N 1/20; C12N 2500/80; C12N 2500/16; C12N 2500/12; C12N 2500/35; C12N 2501/998; C12N 2501/06
USPC 435/101; 435/253.3

(57) ABSTRACT

A specialized culture medium for the promotion of alginate production by stable mucoid Pseudomonas aeruginosa bacterial strains and methods for the production and purification of industrial, commercial, and pharmaceutical grade alginate from bacteriological sources are provided herein. Alginate produced using the media and methods disclosed herein is structurally uniform and substantially free of bacterial cell contaminants, including endotoxin.

### RELATED APPLICATIONS

This application claims priority under 35 U.S.C. §119(e) to U.S. Provisional Application Ser. No. 61/432,762, filed Jan. 14, 2011, the contents of which are incorporated herein by reference in their entirety; and application Ser. No. 13/348,897, filed Jan. 12, 2012.

### FIELD OF THE INVENTION

The presently disclosed subject matter relates to a culture medium and methods for production of alginate from bacterial sources. Specifically, the presently disclosed subject matter relates to a specialized culture medium that promotes alginate production by Pseudomonas aeruginosa (P. aeruginosa) bacteria and methods for production and downstream purification of alginate produced by stable mucoid P. aeruginosa bacterial strains.

### BACKGROUND

Many biopolymers can now be harvested as a renewable resource with minimal impact to the environment. However, reliance on environmental conditions can greatly affect sustainable growth of these resources. Hydrocolloids, such as alginate, are water absorbing polymers that are useful in many industrial and commercial applications. In particular, alginates have been used in a variety of industries and have applications in foods, cosmetics, pharmaceuticals, drug delivery, surgical dressings, wound management, and tissue engineering. Alginates are polysaccharides that are produced by brown seaweeds as well as the Gram negative bacterial genera Pseudomonas and Azotobacter. Alginate is a linear co-polymer of β-D-mannuronate (M) and its C5 epimer α-L-guluronate (G). When P. aeruginosa overproduces alginate, this phenotype is referred to as mucoidy.

The pathways for alginate biosynthesis have been extensively examined in bacteria such as P. aeruginosa. The carbon flow starts with fructose 6-phosphate which is converted to mannose 6-phosphate by the enzyme AlgA. Mannose 6-phosphate is epimerized to mannose 1-phosphate by AlgC, which is converted to GDP-mannose by AlgA. At this point, the conversion from GDP-mannose to GDP-mannuronate by GDP-mannose dehydrogenase, encoded by algD, is the first committed step for alginate biosynthesis. After the mannuronate monomer is polymerized, Alg8 and Alg44 are involved in the polymer transport to the periplasm which is regulated by the second messenger c-di-GMP. A gene, mucR, from outside of the alginate biosynthetic operon generates c-di-GMP near Alg44 to stimulate its activity. Several changes occur to the nascent polymer in the periplasm. Some mannuronic acid residues are converted to the C-5 epimer, guluronate by AlgG. AlgL is an alginate lyase that likely functions in clearing the periplasm of excess alginate, but also influences the length of the alginate polymer. The known periplasmic modification of alginate is acetylation. AlgI, AlgJ, AlgF function to acetylate the mannuronate residues at the 0-2 and/or 0-3 position. Acetylation changes the physical and immunological properties of the alginate polymer and is the main difference from seaweed alginate. The final alginate polymer that has been epimerized, acetylated, and truncated is exported by the alginate porin AlgE.

Alginate production by P. aeruginosa is controlled at the genetic and post-translational levels by genes at several distinct loci. The alternative sigma factor AlgU (also known as AlgT) is responsible for expression of the alginate biosynthetic operon. This operon encodes a cluster of genes for the production, as well as the exportation of alginate. Alginate production is dependent upon the activity status of AlgU. When AlgU is not active, the alginate biosynthetic operon is not expressed and no alginate production occurs. The main negative regulator of AlgU is its cognate anti-sigma factor MucA. MucA is an inner membrane protein with its N-terminus at the cytoplasmic side that sequesters AlgU, rendering it inactive. The prototypic strain PAO1 has minimal AlgU activity resulting in a non-mucoid phenotype. AlgU is activated when mucA is mutated or when MucA is proteolytically cleaved. MucB protects the periplasmic C-terminus of MucA from such cleavages. The sequential degradation of MucA by proteases follows the scheme of regulated intramembrane proteolysis (RIP). During RIP, MucA is degraded by the site 1 protease AlgW followed by the site 2 protease MucP. AlgW protease can be activated by a small envelope protein known as MucE, which has a unique C-terminal sequence of WVF. Activated AlgW in strain VE2 is due to the accumulated WVF signal of MucE. When AlgW is activated by MucE, AlgW will cleave the C-terminus of MucA. After AlgW cleavage of MucA, further degradation of MucA will occur by MucP. This sequential proteolysis of MucA results in active AlgU. Active AlgU initiates expression of the alginate biosynthesis machinery resulting in a mucoid phenotype. Several strains of P. aeruginosa have been genetically engineered to be stable mucoid strains that produce greater amounts of alginate, compared to wild-type P. aeruginosa, including strains VE2 and VE19 (Qiu, D. et al., Regulated proteolysis controls mucoid conversion in Pseudomonas aeruginosa, Proc. Natl. Acad. Sci. USA 104:8107-12 (2007)), VE19algW (Damron, F. et al., Pseudomonas aeruginosa MucD regulates alginate pathway through activation of MucA degradation via MucP proteolytic activity, J. Bacteriol. 193:286-291 (2011)), and VE13 (Damron, F. et al., The Pseudomonas aeruginosa sensor kinase KinB negatively controls alginate production through AlgW-dependent MucA proteolysis, J. Bacteriol. 191:2285-95 (2009)).

The world's supply of alginate is presently harvested from brown seaweeds. However, the composition of seaweed alginate is not uniform, due to environmental conditions and other factors. Seasonal inconsistency, batch inconsistency, and the 15 to 20 processing steps necessary to obtain the final alginate product all increase the overall variability, chemical waste, production time and cost associated with producing alginate from seaweed.

The need persists for improved materials and methods for the production and purification of alginate from bacterial sources, such as P. aeruginosa.

### SUMMARY OF THE INVENTION

The present inventors have now developed a specialized culture medium and methods of use for the production of alginate from mucoid strains of P. aeruginosa. The presently disclosed culture medium and methods result in consistently high yields of commercial grade alginate polymer suitable for use in a variety of applications. The present methods provide uniform and structurally consistent alginate, while reducing production time, variability, chemical waste, and cost.

In one embodiment, a culture medium for the promotion of alginate production by P. aeruginosa bacterial cells belonging to a strain having a stable mucoid phenotype is provided, the culture medium comprising a nitrogen source; K₂SO₄; MgCl₂; and from about 5% (v/v) to about 14% (v/v) glycerol.

In another embodiment, a method for producing alginate from P. aeruginosa bacterial cells belonging to a strain having a stable mucoid phenotype is provided, the method comprising: (a) growing P. aeruginosa bacterial cells in a liquid culture medium, wherein the bacterial cells secrete alginate in the liquid culture medium; (b) dehydrating the liquid culture medium with ethanol to provide a first dehydrated alginate fraction; (c) filtering the first dehydrated alginate fraction through a Dutch weave wire mesh filter to collect alginate; (d) resuspending the alginate collected in step (c) in ethanol; and (e) filtering the alginate resuspended in step (d) through a Dutch weave wire mesh filter to collect washed alginate.

These and other objects, features, embodiments, and advantages will become apparent to those of ordinary skill in the art from a reading of the following detailed description and the appended claims.

### BRIEF DESCRIPTION OF THE DRAWINGS

FIG. 1(A) shows the β-D-mannuronate (M) and α-L-guluronate (G) subunits that comprise bacterial alginate. FIG. 1(B) shows the chain conformation of bacterial alginate with a block structure of GMMG, with an acetyl groups at the M residues. FIG. 1(C) shows an exemplary block distribution of alginate.

FIG. 2(A) shows a top view of Dutch weave wire mesh. FIG. 2(B) shows a cross-sectional view of Dutch weave wire mesh.

FIG. 3(A) shows the orthogonal design for preparation of media for comparison. Flasks 1-16 were prepared with components indicated in their respective rows. Experiments were conducted in a 500 ml flask containing a volume of 70 ml media. FIG. 3(B) shows the calculations for the orthogonal design for media comparison.

FIG. 4(A) shows the effect of medium composition on the growth of P. aeruginosa strain VE2, as measured by OD₆₀₀. FIG. 4(B) shows the effect of medium composition on alginate production by VE2, as measured in alginate g/L.

FIG. 5 shows the results of alginate production in g/L and growth (OD₆₀₀) for each of flasks 1-16 prepared according to FIG. 3.

FIG. 6 shows the experimental design used to test the effect of glycerol concentration on alginate production by P. aeruginosa strain VE2. Flasks were prepared in duplicate for each concentration of glycerol.

FIG. 7 shows the results of glycerol concentration on alginate production in g/L and growth (OD₆₀₀) for each of flasks 1-14 prepared according to FIG. 6.

FIG. 8(A) shows the experimental design used to test the effect of various carbon sources on alginate production by P. aeruginosa strain VE2. FIG. 8(B) shows the effect of the carbon source on alginate production over time, in g/L. FIG. 8(C) shows the alginate production (g/L) normalized for growth as measured by OD₆₀₀ at 72 hours.

FIG. 9 shows the comparison of alginate production (g/L) by VE2 in two different media over time: Pseudomonas Isolation Broth (PIB), and PIBS custom media, also known as Alginate Super Media (ASM).

FIG. 10(A) shows the viscosity vs. shear rate for alginate produced according to the instant methods. Results show as shear rate increases, viscosity decreases. The 68 hour and 72 hour samples are approximately 10 times as viscous as the 48 hour sample, suggesting that the longer period of growth (68 and 72 hours, respectively) in a bioreactor increases the molecular weight of alginate polymer. FIG. 10(B) shows the shear stress vs. shear rate for alginate produced according to the instant methods. Results show as shear rate increases, shear stress increases. FIG. 10(C) shows the viscosity vs. shear stress for alginate produced according to the instant methods. Results show as stress increases, viscosity decreases. A slight yield stress was observed. At 0.01 reciprocal seconds, the stress for the 68 and 72 hour samples is estimated to be about 8 Pa.

FIG. 11 shows the relationship between physical characteristics and temperature of alginate as produced according to the instant methods. Results show the shear viscosity (A), stress shear rate (B), and stress viscosity (C) of alginate dispersions harvested from the growth of strain VE2 in ASM are independent of temperature, suggesting that a high speed double planetary mixer as detailed in FIGS. 15-17 is suitable for industrial processing of bacterial alginate.

FIG. 12 is a flow chart showing the Phase I processing steps for the production of alginate from P. aeruginosa.

FIG. 13 is a flow chart showing the Phase II processing steps for the production of alginate from P. aeruginosa.

FIG. 14 is a flow chart showing the Phase III processing steps for the production of alginate from P. aeruginosa.

FIG. 15 shows an exemplary design for the Phase I (also called Step 1) large-scale production of alginate.

FIG. 16 shows an exemplary design for the Phase II (also called Step 2) large-scale production of alginate.

FIG. 17 shows an exemplary design for the Phase III (also called Step 3) large-scale production of alginate.

FIG. 18 shows HPLC analysis of alginate.

FIG. 19 shows NMR analysis of alginate.

### DETAILED DESCRIPTION OF THE INVENTION

The details of one or more embodiments of the presently-disclosed subject matter are set forth in this document. Modifications to embodiments described in this document, and other embodiments, will be evident to those of ordinary skill in the art after a study of the information provided in this document.

While the following terms are believed to be well understood by one of ordinary skill in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

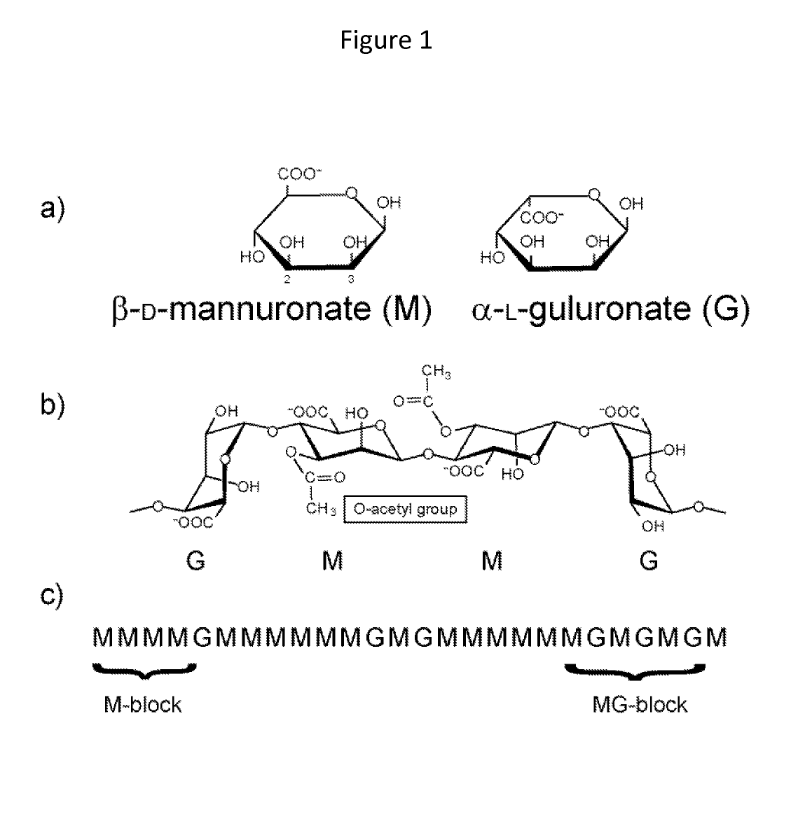

The term “alginate” refers to a linear copolymer of 1,4 linked β-D-mannuronate (M) and its C5 epimer α-L-guluronate (G). Alginate can be obtained from seaweed as well as bacterial sources, such as P. aeruginosa and Azotobacter. Alginate obtained from seaweed is characterized by a structure and composition that is variable between growing seasons, which in the past has limited alginate's use in medical and pharmaceutical applications. Bacterial alginate differs from seaweed alginate by the O-acetylation at the 2 and/or 3 position of mannuronate. Alginate biopolymer consists of variable length M blocks and MG blocks. See FIG. 1.

A strain of P. aeruginosa is considered a “stable mucoid strain” if, after repeated passage of a single colony for two weeks on daily basis, the phenotype of the single colony remains mucoid (alginate-overproducing) on a Pseudomonas isolation agar (PIA) plate, and greater than 99% of the colonies derived from the single colony on each passage also remain mucoid on a PIA plate.

Strain VE2 is a stable mucoid variant of P. aeruginosa isolated from a mutational screen of the prototypic strain of P. aeruginosa PAO1, using a mariner transposon called pFAC. This transposon, which has a gentamicin resistance marker, is capable of inserting itself into any TA dinucleotide in the genome of P. aeruginosa. VE2 is a mutant that displays a stable production of copious amounts of alginate on PIA media. The insertion site that was mapped before mucE (PA4033) provides an artificial signal for the constitutive expression of mucE. The induction of MucE is sufficient to activate a protease called AlgW for the initiation of the MucA degradation, thus activating the production of alginate in strain VE2. This strain also displays a stable characteristic of alginate production in the Pseudomonas isolation broth (PIB). See Qiu, D. et al., Regulated proteolysis controls mucoid conversion in Pseudomonas aeruginosa, Proc. Natl. Acad. Sci. USA 104:8107-12 (2007).

Strain 581 is a stable mucoid variant of P. aeruginosa. Strain 581 was originally isolated following in vitro incubation of the non-mucoid PAO strain with phage E79. The strain carries undefined muc mutation(s) (designated the muc-25 variant). The muc-25 mutation was identified as a single base deletion at T180 of mucA, leading to creation of a premature stop codon (TGA) at position 285. The resulting frameshift encoded a truncated polypeptide of 94 aa (MucA25) containing the N-terminal 59 aa of MucA. While not desiring to be bound by theory, it is believed that the degradation of N-terminus of MucA25 may be attributed to the increased activity of the ClpXP protease complex in strain 581, thus causing the stable production of alginate. Strain 581 also can stably produce alginate in PIB. See Qiu, D. et al., ClpXP proteases positively regulate alginate overexpression and mucoid conversion in Pseudomonas aeruginosa, Microbiology 154:2119-30 (2008).

In certain embodiments, the alginate produced by P. aeruginosa strains such as VE2, 581, or other strains genetically modified to over-produce alginate is alternatively termed genetically engineered alginate, or GEA™.

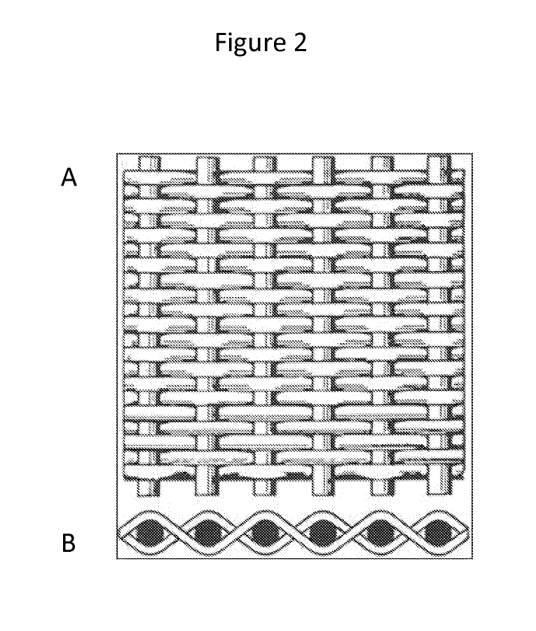

The term “Dutch weave wire mesh filter” refers to a filter made from wire mesh which is woven with a larger diameter wire in the warp direction and a comparatively smaller diameter wire in the shute direction. See FIG. 2. Dutch weave wire mesh filters are particularly useful in the methods of the present invention because they allow alginate fibers to be removed from the filter without dislodging pieces of the filter into the sample, which affects sample purity. Since the size of P. aeruginosa is generally between 2-5 μm, the use of certain pore size Dutch weave wire mesh filters allows the passage of free floating bacterial cells while retaining the alginate fibers on the filter. In some embodiments, Dutch weave wire mesh filters suitable for use in the instant methods have a pore size (or space) between wires of from about 2 μm to about 20 μm, from about 5 μm to about 15 μm, from about 7 μm to about 12 μm, from about 8 μm to about 11 μm, or about 10 μm. In a very specific embodiment, Dutch weave wire mesh filters suitable for use in the instant methods have a pore size (or space) between wires of about 10 μm. Dutch weave wire mesh is available from a variety of commercial sources, including Dorstener Wire Tech (Spring, Tex.).

### Culture Medium

The instant inventors have developed a specialized culture medium for the promotion of alginate production by stable mucoid strains of P. aeruginosa. The nitrogen source, salts, and carbon source and concentrations thereof present in the culture medium promote production of alginate and produce a surprisingly superior yield of alginate over time, as compared with traditional Pseudomonas media, including Pseudomonas Isolation Agar (PIA), Pseudomonas Isolation Broth (PIB), PIB supplemented with agar, or Lennox Broth (LB) agar.

In one embodiment, the culture medium (also called “Alginate Super Medium,” or “ASM”) comprises sterile water and a nitrogen source, K₂SO₄, MgCl₂, and glycerol.

### Nitrogen Source

In one embodiment, the nitrogen source is selected from the group consisting of bactopeptone, pancreatic digest of gelatin, and combinations thereof. In a specific embodiment, the concentration of the nitrogen source is from about 0.5% to about 15%, from about 1% to about 10%, from about 1% to about 5%, from about 1% to about 3%, or about 2% (w/v). In a specific embodiment, the nitrogen source comprises about 1% (w/v) bactopeptone and about 1% (w/v) pancreatic digest of gelatin, for a total of about 2% (w/v) of a nitrogen source.

### Salts

In one embodiment, the presently disclosed culture medium comprises salts selected from the group consisting of K₂SO₄ and MgCl₂. In a specific embodiment, the medium comprises from about 0.5% to about 3%, from about 1% to about 2.5%, from about 1.5% to about 2.5%, or about 2% (w/v) K₂SO₄.

In another embodiment, the presently disclosed culture medium comprises from about 0.05 to about 0.5%, from about 0.10 to about 0.5%, from about 0.25 to about 0.5%, 0.75% to about 0.5%, or about 0.5% (w/v) MgCl₂.

### Glycerol

Glycerol is present in the culture medium as a carbon source. In one embodiment, the culture medium disclosed herein comprises from about 5% to about 14%, from about 5% to about 12%, from about 7% to about 12%, from about 8% to about 12%, from about 9% to about 11%, or about 10% (v/v) glycerol.

### Triclosan

Triclosan, or 5-chloro-2-(2,4-dichlorophenoxyl)phenol (also called Irgasan®), is an antibacterial and antifungal agent often added to PIB or PIA. Moreover, P. aeruginosa cultures supplemented with triclosan have been shown to promote the production of alginate. Hence, adding triclosan to the culture medium serves to prevent contamination by microorganisms susceptible to triclosan, while also promoting alginate production. Thus, in certain embodiments, the culture medium further comprises triclosan. In a specific embodiment, the culture medium comprises about 25 mg/liter triclosan.

In a very specific embodiment, the ASM culture medium comprises sterile water and about 1% (w/v) bactopeptone; about 1% (w/v) pancreatic digest of gelatin; about 2% (w/v) K₂SO₄; about 0.5% (w/v) MgCl₂; and about 10% (v/v) glycerol. In a more specific embodiment, the culture medium further comprises about 25 mg/liter triclosan.

The culture media disclosed herein are particularly useful for the culture of stable mucoid strains of P. aeruginosa strains, such as VE2 and 581 and have been shown to promote alginate production by such mucoid strains.

Methods for Production of Alginate from P. aeruginosa

Production of biopolymers such as alginate from bacteriological sources requires the removal of bacterial cells from the end product. Pressure filtration and centrifugation are commonly used to remove bacterial cells; however these methods require application of high forces to the product, which is directly correlated with viscosity, which impedes the separation of bacterial cells from alginate. Consequently, solutions are often diluted as needed in order to facilitate separation of bacterial cells from viscous samples. However, harvesting alginate from diluted samples is more difficult because more ethanol will be required to precipitate the alginate fibers. Thus, harvesting alginate from bacteriological sources using methods currently practiced in the art is time consuming, requires excess energy consumption, and increases the number of processing steps and materials required to recover the alginate product.

The present inventors have developed methods for the production and purification of alginate from stable mucoid stains of P. aeruginosa which overcome the current pitfalls while providing a high purity alginate product free of bacterial cell contamination.

### Phase I

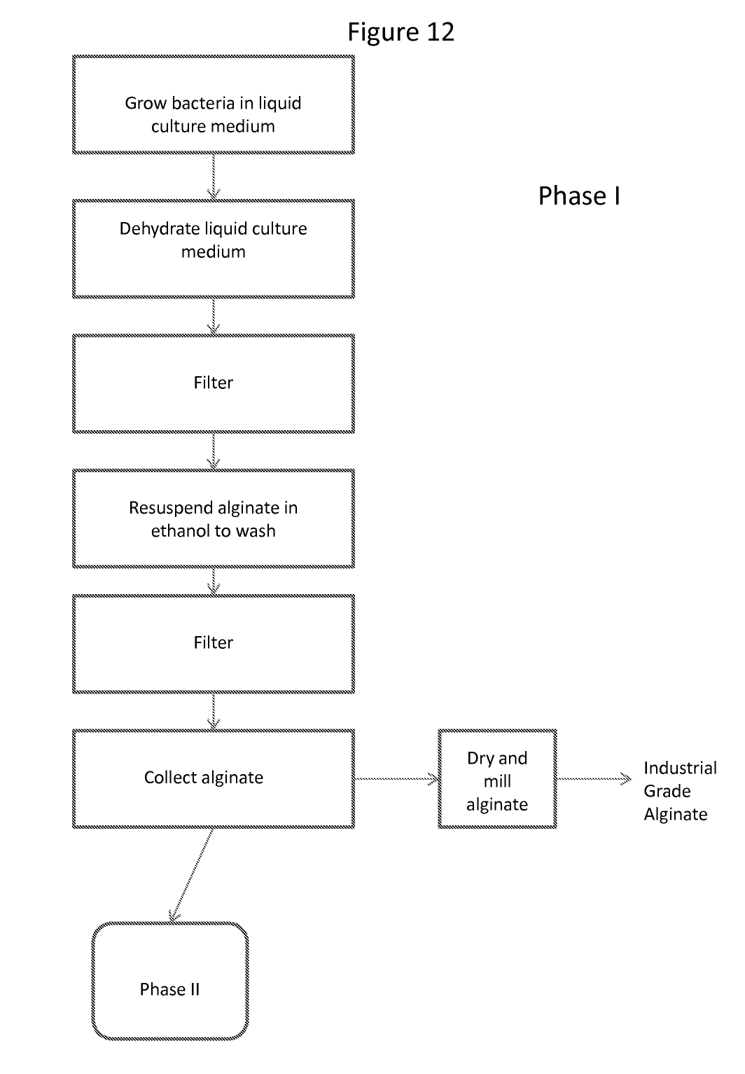

Phase I of production and purification provides an alginate product suitable for use in a variety of industrial applications. In this embodiment, a method for producing alginate from P. aeruginosa bacterial cells belonging to a strain having a stable mucoid phenotype is provided, the method comprising: (a) growing P. aeruginosa bacterial cells in a liquid culture medium, wherein the bacterial cells secrete alginate in the liquid culture medium; (b) dehydrating the liquid culture medium with ethanol to provide a first dehydrated alginate fraction; (c) filtering the first dehydrated alginate fraction through a Dutch weave wire mesh filter to collect alginate; (d) resuspending the alginate collected in step (c) in ethanol; and (e) filtering the alginate resuspended in step (d) through a Dutch weave wire mesh filter to collect washed alginate. FIG. 12 shows a schematic representation of Phase I.

In one embodiment, the ethanol employed in the instant methods is denatured ethanol. In another embodiment, the ethanol has a concentration of greater than about 70%, greater than about 85%, greater than about 90%, greater than about 95%, or about 100%. In another embodiment, the ethanol has a concentration of from about 70% to about 100%, from about 85% to about 100%, from about 90% to about 100%, or from about 95% to about 100%.

Other reagents are also suitable for use in the dehydration and resuspension/washing steps of the purification process. Although 85-100% denatured ethanol is particularly useful, other alcohols, such as isopropanol or 2-propanol, are also suitable for use in the presently disclosed processes. Generally, the greater the percentage of alcohol, the greater the percentage of alginate recovered from the solution.

P. aeruginosa cultures can be grown using a variety of techniques known to the skilled artisan. In one embodiment, the cultures are grown overnight in liquid culture media (about 16 hours) at 37° C. in a shaking incubator. In another embodiment, cultures are grown in liquid culture media in a bioreactor, which is a culture vessel of certain volume (generally 3-5 liters) with controlled dissolved oxygen, pH, and temperature. In most cases, a medium reservoir can be attached to the vessel to supply fresh medium for culture growth.

In one embodiment, the culture medium used to grow the P. aeruginosa bacterial cells is Alginate Super Medium (ASM), as disclosed herein. In a specific embodiment, the ASM comprises from about 0.5% (w/v) to about 15% (w/v) of a nitrogen source selected from the group consisting of bactopeptone and pancreatic digest of gelatin and combinations thereof; from about 0.5% (w/v) to about 3% (w/v) K₂SO₄; from about 0.05% (w/v) to about 0.5% (w/v) MgCl₂; and from about 5% (v/v) to about 14% (v/v) glycerol. In a more specific embodiment, the ASM culture medium comprises about 1% (w/v) bactopeptone; about 1% (w/v) pancreatic digest of gelatin; about 2% (w/v) K₂SO₄; about 0.5% (w/v) MgCl₂; and about 10% (v/v) glycerol. In another embodiment, the ASM further comprises about 25 mg/liter triclosan.

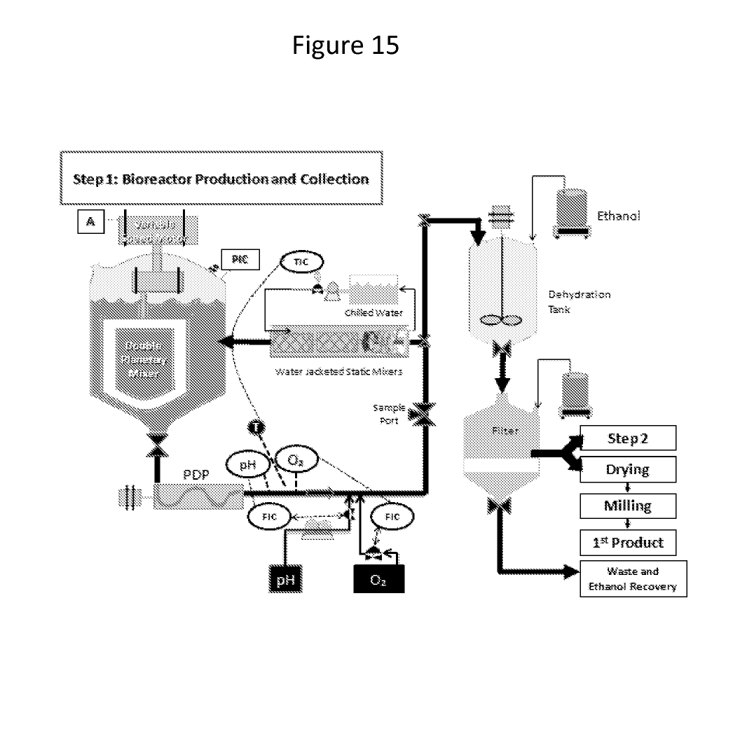

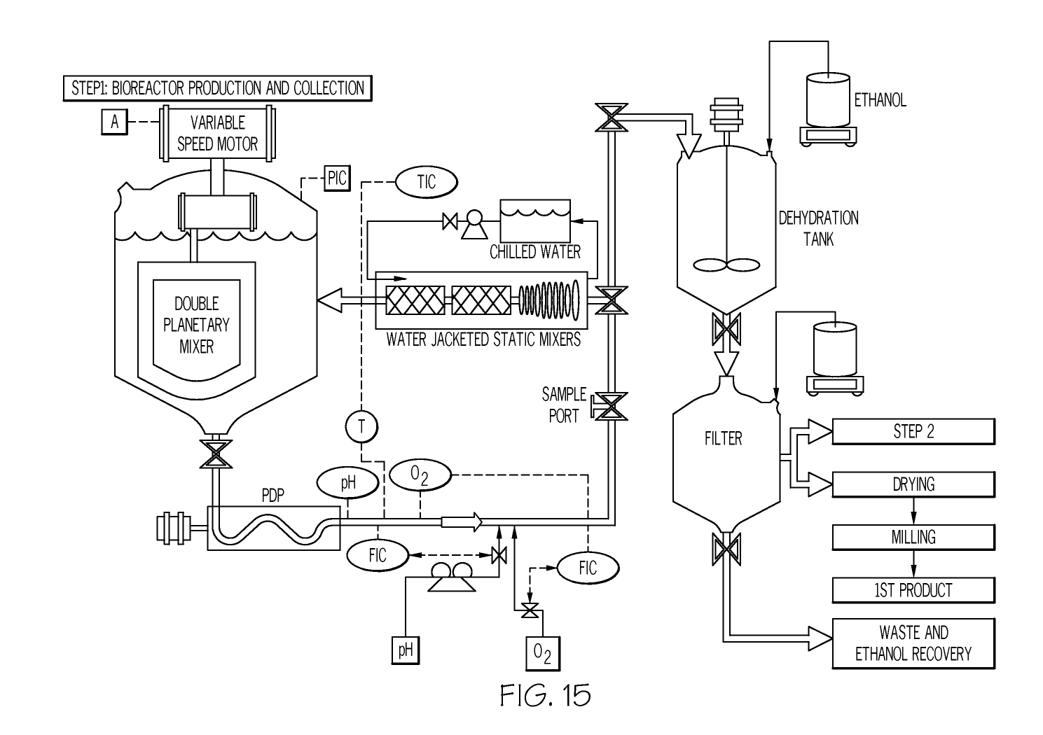

Phase I processing may also be scaled up for large-scale production and purification of alginate from bacteriological sources. FIG. 15 shows an exemplary design for the large-scale production of alginate.

At this point in the production process, alginate collected in step (e) can optionally be dried and milled for use in industrial applications.

In another embodiment, alginate collected in step (e) of Phase I proceeds to Phase II further for processing to provide a higher purity alginate product.

### Phase II

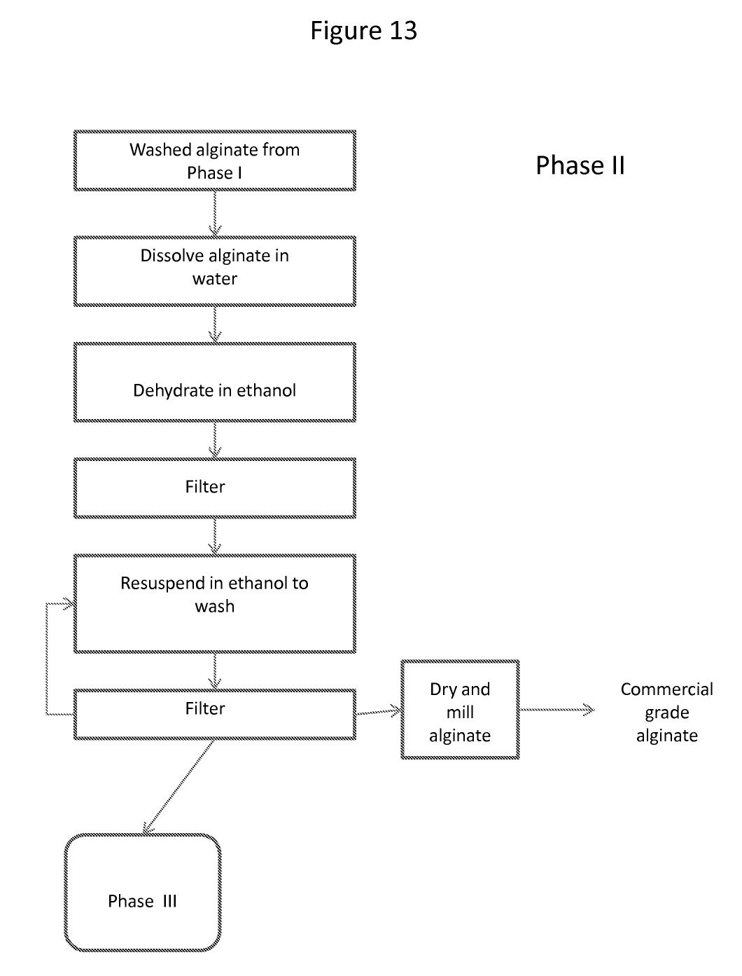

If a higher purity alginate product is desired, the washed alginate collected from Phase I proceeds to Phase II of the purification process. Thus, in another embodiment, the method further comprises step (f), rehydrating the washed alginate collected in step (e) by solubilizing the washed alginate in water to provide a rehydrated alginate solution; (g) dehydrating the rehydrated alginate solution with ethanol to provide a second dehydrated alginate fraction; (h) filtering the second dehydrated alginate fraction of step (g) through a Dutch weave wire mesh filter to collect alginate; (i) resuspending the alginate collected in step (h) in ethanol; and (j) filtering the alginate resuspended in step (i) through a Dutch weave wire mesh filter to collect alginate. FIG. 13 shows a schematic representation of Phase II. In one embodiment, washing steps (i) and (j) can be repeated to increase the purity of the washed alginate. For example, in a specific embodiment, steps (i) and (j) together are repeated one, two, three, or more times before proceeding to drying or further processing.

In one embodiment, the ethanol employed in the instant methods is denatured ethanol. In another embodiment, the ethanol has a concentration of greater than about 70%, greater than about 85%, greater than about 90%, greater than about 95%, or about 100%. In another embodiment, the ethanol has a concentration of from about 70% to about 100%, from about 85% to about 100%, from about 90% to about 100%, or from about 95% to about 100%.

Other reagents are also suitable for use in the dehydration and resuspension/washing steps of the purification process. Although 85-100% denatured ethanol is particularly useful, other alcohols, such as isopropanol or 2-propanol, are also suitable for use in the presently disclosed processes. Generally, the greater the percentage of alcohol in the reagent, the greater the percentage alginate recovered from the solution.

At this point in Phase II of the process, alginate collected in step (j) can optionally be dried and milled for use in industrial applications. The drying step can be accomplished using a variety of techniques known to those skilled in the art. In one embodiment, the alginate is dried using a vacuum oven. Other suitable drying methods include, for example, mechanical removal of the moisture followed by heating the samples or air drying the samples to remove moisture.

Alginate recovered after Phase II processing is suitable for use in a variety of commercial applications, including use in the food industry as an additive or as an ingredient in personal care products. In one embodiment, alginate recovered after Phase II processing has a purity of greater than about 50% compared to seaweed alginate, removing a majority of cell debris and pigmentation associated with bacterial alginate. In one embodiment, alginate processed according to Phase II methods as set forth herein is substantially free of bacterial cell contaminants and endotoxin, without a need to centrifuge the sample.

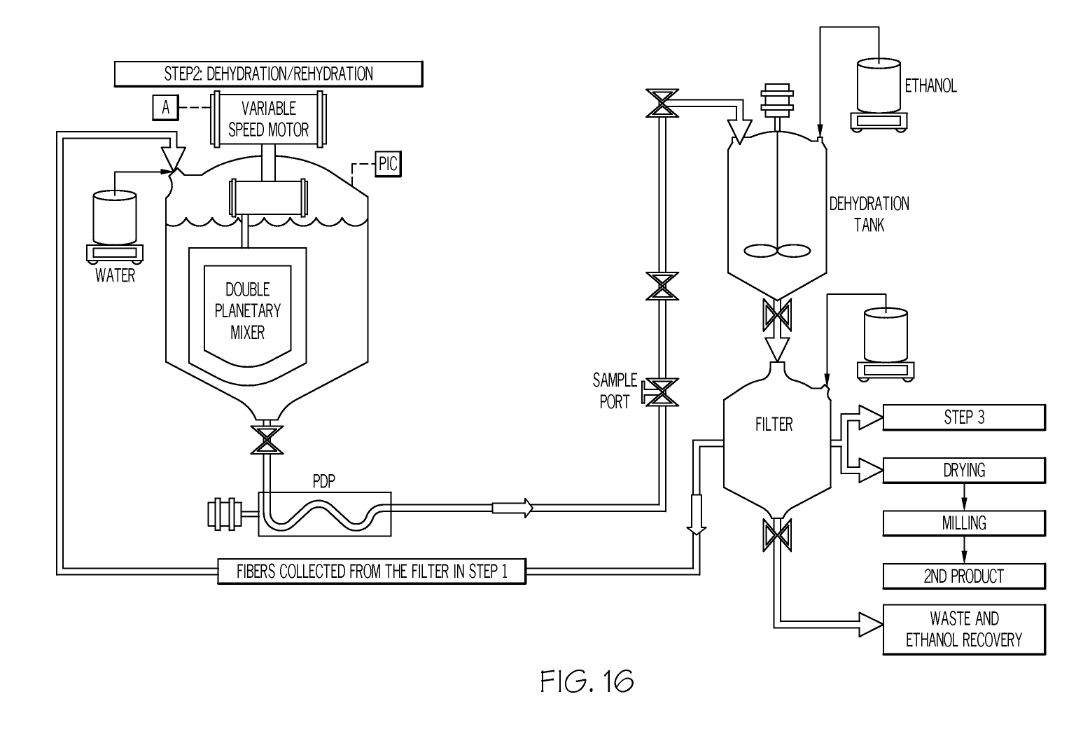

Phase II processing may also be scaled up for large-scale production and purification of alginate from bacteriological sources. FIG. 16 shows an exemplary design for the large-scale production of alginate.

In another embodiment, alginate collected in step (j) of Phase II proceeds to Phase III for further processing to provide a higher purity alginate product.

### Phase III

If a still higher purity alginate product is desired, the washed alginate collected from Phase II proceeds to Phase III for further processing. Thus, in another embodiment, the method further comprises the steps of (k) rehydrating the alginate collected in step (j) by solubilizing the washed alginate completely in water to provide a rehydrated alginate solution; (l) passing the rehydrated alginate solution of step (k) through an ion exchange column; (m) washing the ion exchange column with at least one NaCl wash solution having a concentration of from about 0.2 M to about 3 M and collecting eluted wash solution; (n) concentrating the eluted wash solution by passing the eluted wash solution through a molecular sieve filter; (o) dehydrating the eluted wash solution concentrated in step (n) with ethanol to provide a second dehydrated alginate fraction; and (p) filtering the second dehydrated alginate fraction through a Dutch weave wire mesh filter to collect alginate.

In one embodiment, the ethanol employed in the instant methods is denatured ethanol. In another embodiment, the ethanol has a concentration of greater than about 70%, greater than about 85%, greater than about 90%, greater than about 95%, or about 100%. In another embodiment, the ethanol has a concentration of from about 70% to about 100%, from about 85% to about 100%, from about 90% to about 100%, or from about 95% to about 100%.

Other reagents are also suitable for use in the dehydration and resuspension/washing steps of the purification process. Although 85-100% denatured ethanol is particularly useful, other alcohols, such as isopropanol or 2-propanol, are also suitable for use in the presently disclosed processes. Generally, the greater the percentage of alcohol, the greater the percentage of alginate recovered from the solution.

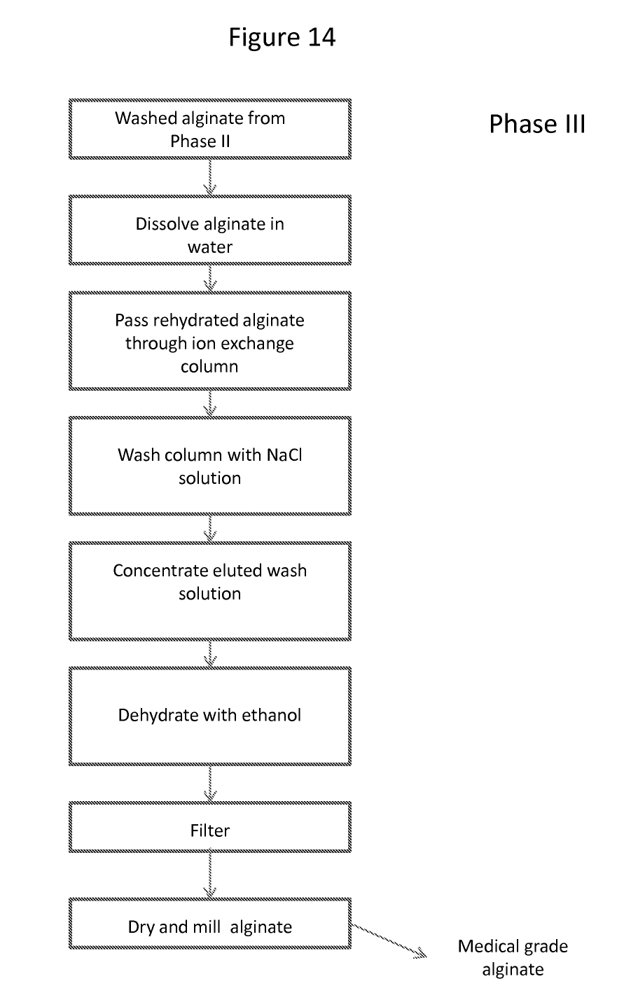

In a further embodiment, the alginate is then dried and milled to provide a purified alginate product. As noted supra, the drying step can be accomplished using a variety of techniques known to those skilled in the art. In one embodiment, the alginate is dried using a vacuum oven. Other suitable drying methods include, for example, mechanical removal of the moisture followed by heating the samples or air drying the samples to remove moisture. FIG. 14 shows a schematic representation of Phase III.

Alginate recovered via ion exchange chromatography after Phase III processing is suitable for use in a variety of medical or pharmaceutical applications. In one embodiment, alginate recovered via ion exchange chromatography has a purity of greater than about 95% compared to seaweed alginate, removing a majority of cell debris and pigmentation associated with bacterial alginate. In one embodiment, alginate processed according to Phase III methods as set forth herein is substantially free of bacterial cell contaminants and endotoxin, without a need to centrifuge the sample.

In another embodiment, the alginate is comprised of about 70% mannuronate and about 30% guluronate.

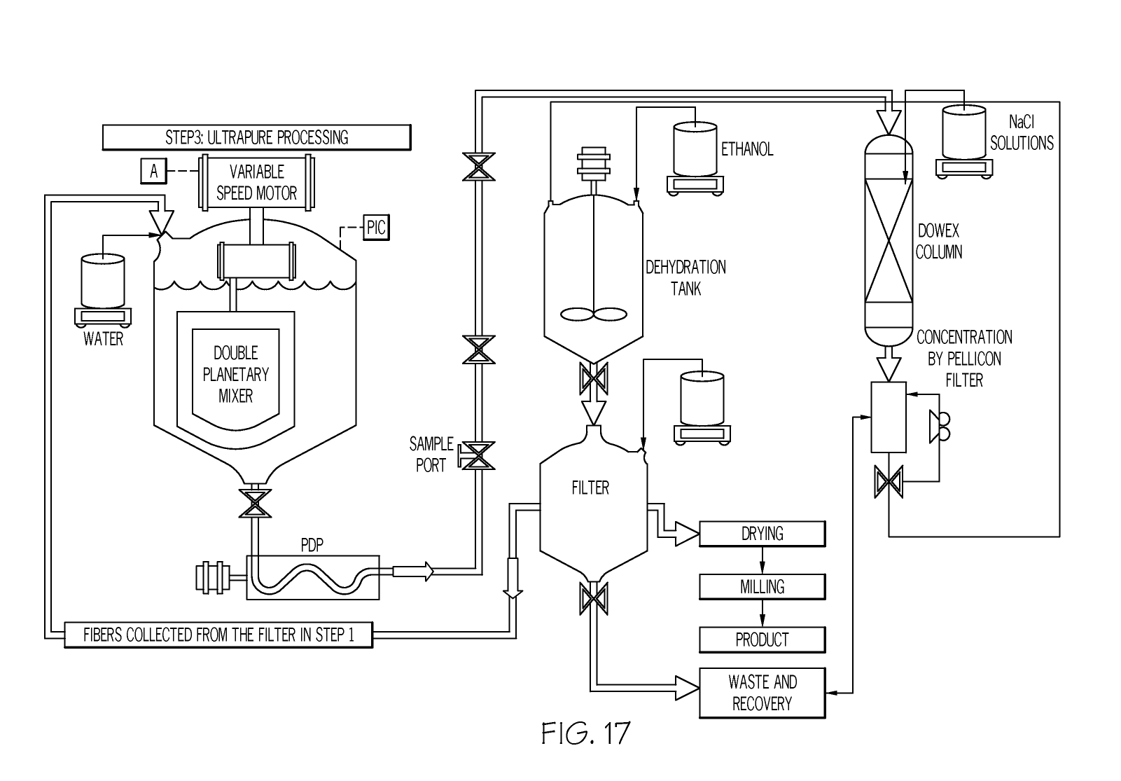

Phase III processing may also be scaled up for large-scale production and purification of alginate from bacteriological sources. FIG. 17 shows an exemplary design for the large-scale production of alginate.

### EXAMPLES

The following examples are given by way of illustration and are not intended to limit the scope of the present invention.

### Example 1

### Effect of Nitrogen, Carbon, and Salts on Alginate Production

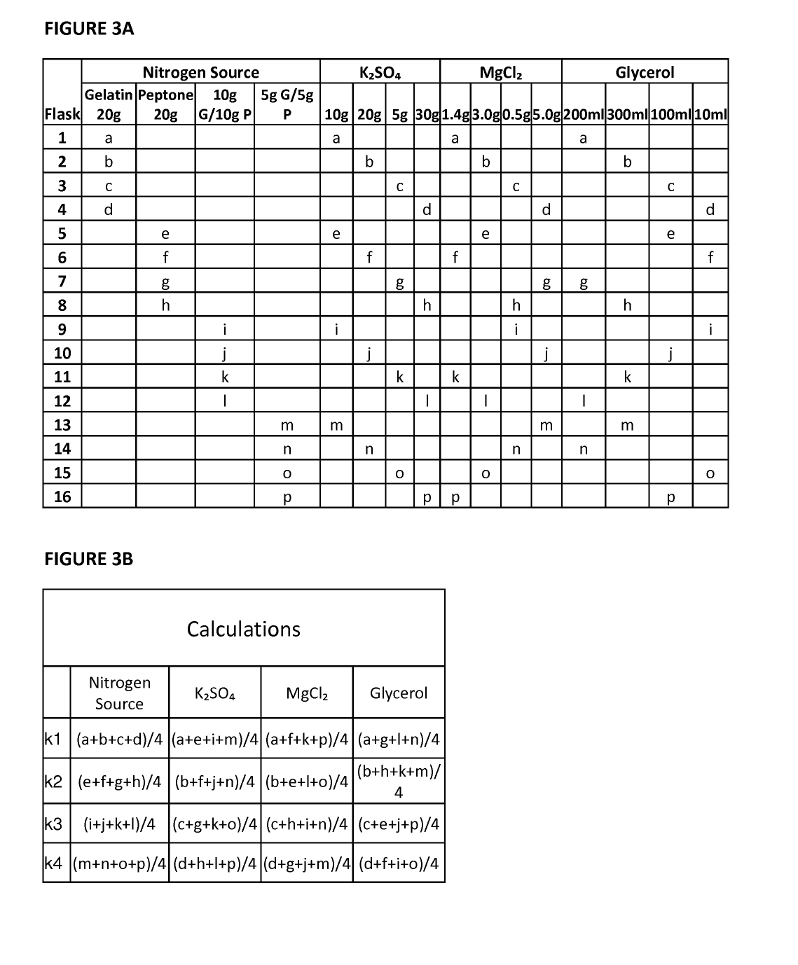

VE2 from frozen stocks was grown on PIA and then an isolated on PIA. A single colony was transferred and grown at 37° C. and 150 rpm overnight in 250 ml PIB media. Stock solutions were made of 100 g/L bactopeptone, 100 g/L pancreatic digest of gelatin, 200 g/L K₂SO₄ (dissolve over heat), 50 g/L MgCl₂, 50% glycerol, and 2.5 mg/ml triclosan and autoclaved. Flasks were prepared as shown in FIGS. 3(A) and 3(B) with exactly 70 ml final total volume. (Example: Flask 1 contains 14 ml pancreatic digest of gelatin stock, 3.5 ml K₂SO₄ stock, 1.96 ml MgCl₂, 28 ml glycerol, 700 μl triclosan, and 22.54 ml H₂O.) Flasks were then inoculated with 6 ml of the overnight culture and placed on a 150 rpm shaker at 37° C., and incubated for to 72 hours. Samples (6 ml) were taken every 12 hours. 1 ml was used for OD₆₀₀ and 5 ml was dehydrated with 100% ethanol. Crude samples were then analyzed for their content in the mannuronic acid curve and corrected for OD₆₀₀ (estimation of cell density) obtained by a spectrophotometer. A 5 ml sample was dehydrated with 15 ml 100% ethanol to obtain a wet fiber weight. Fibers were collected carefully and pressed to remove excess ethanol.

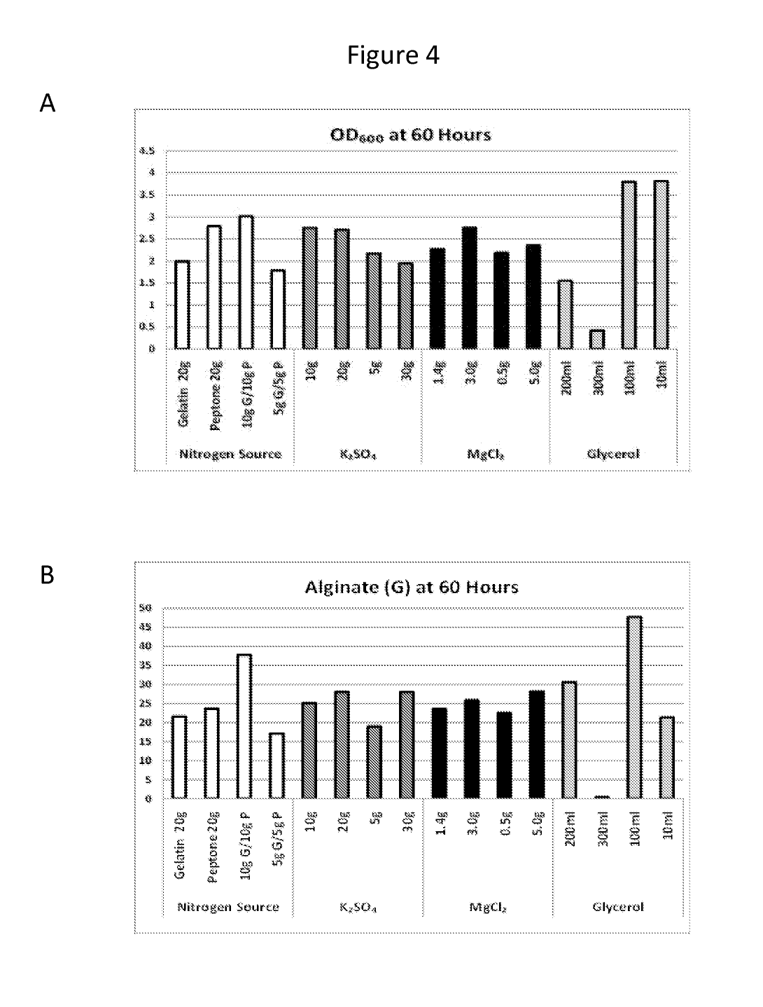

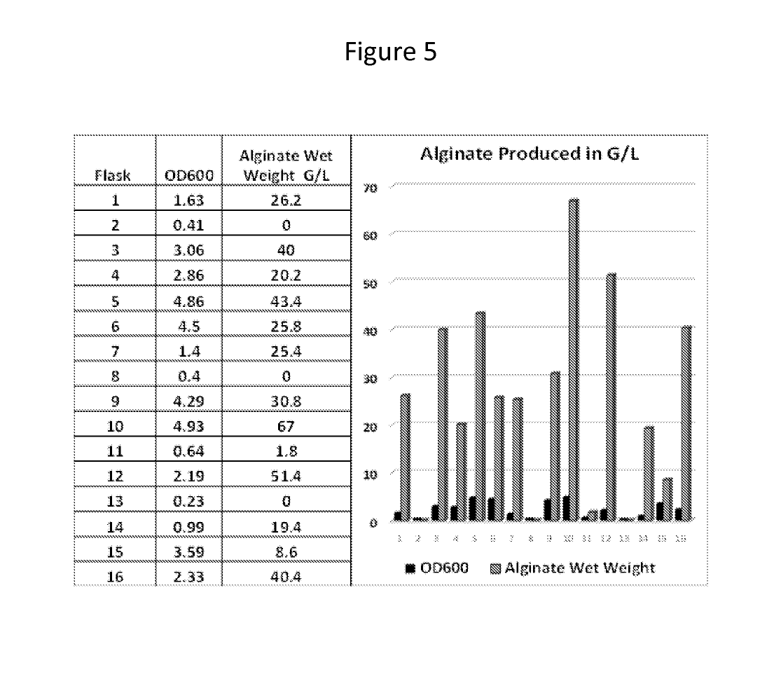

The results of the salt and nutrient comparison are shown in FIGS. 4 and 5. It was observed that mixing equal amounts of gelatin (4% w/v) and bactopeptone (4% w/v) gives a higher production yield of alginate compared to the single component alone (8% w/v). Concentration of about 10% v/v glycerol in the medium provides the highest yield of alginate (67 g/liter), as exemplified in Flask 10.

### Example 2

### Effect of Glycerol Concentration on Alginate Production

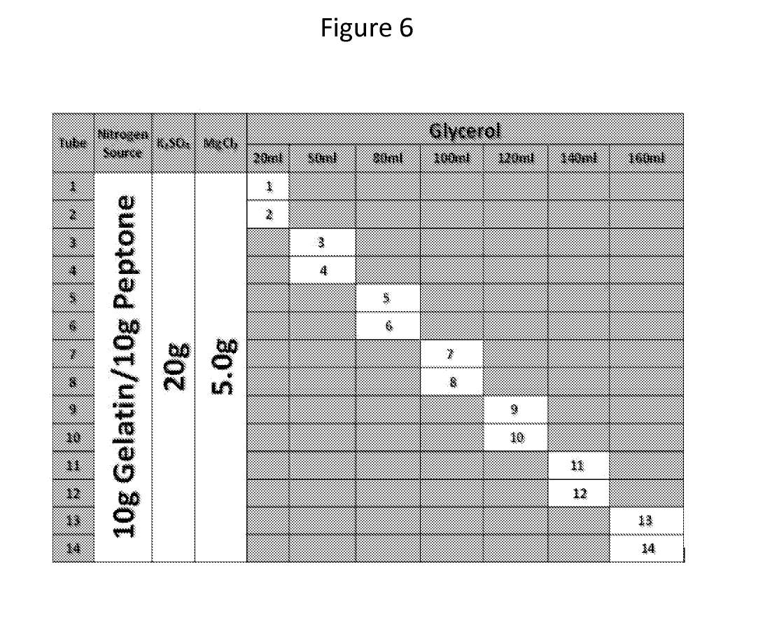

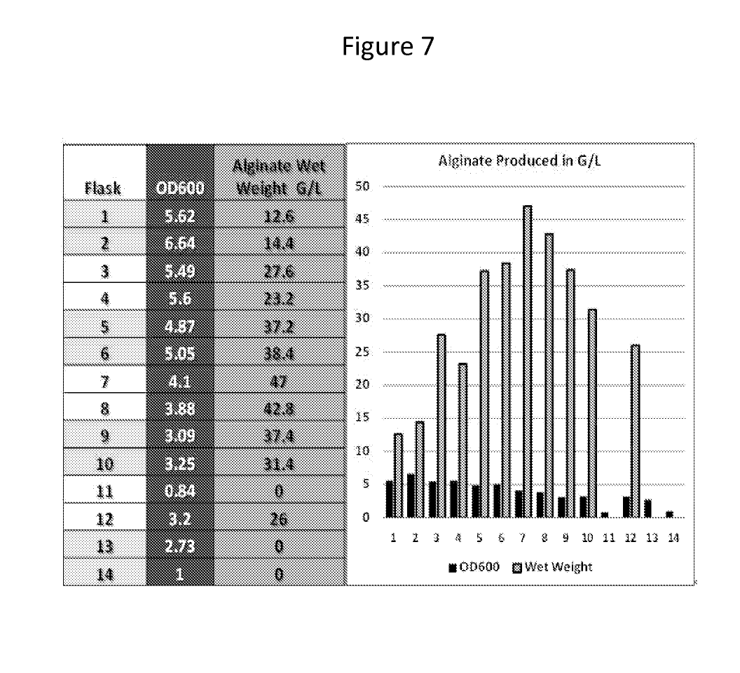

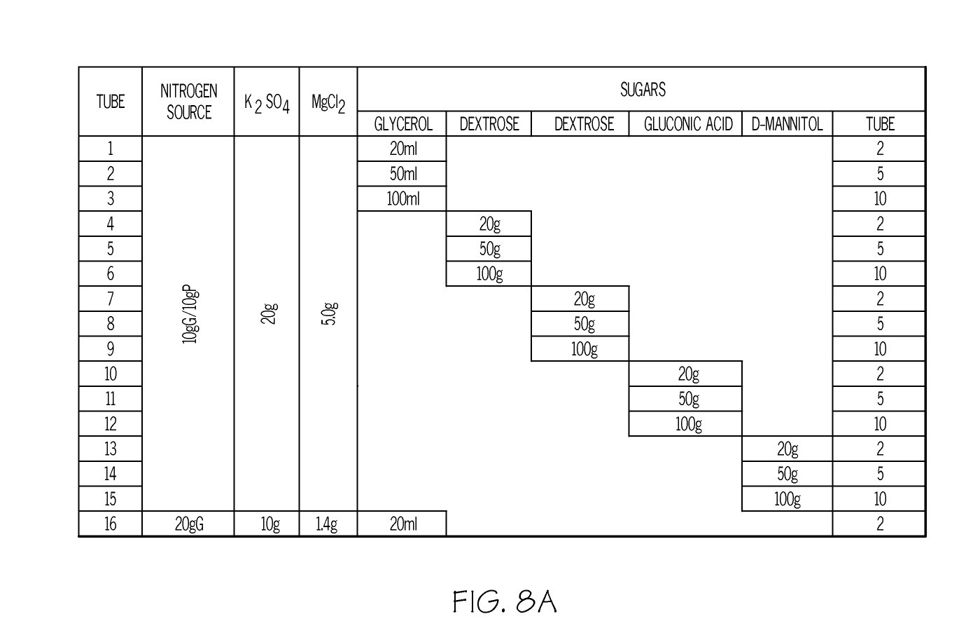

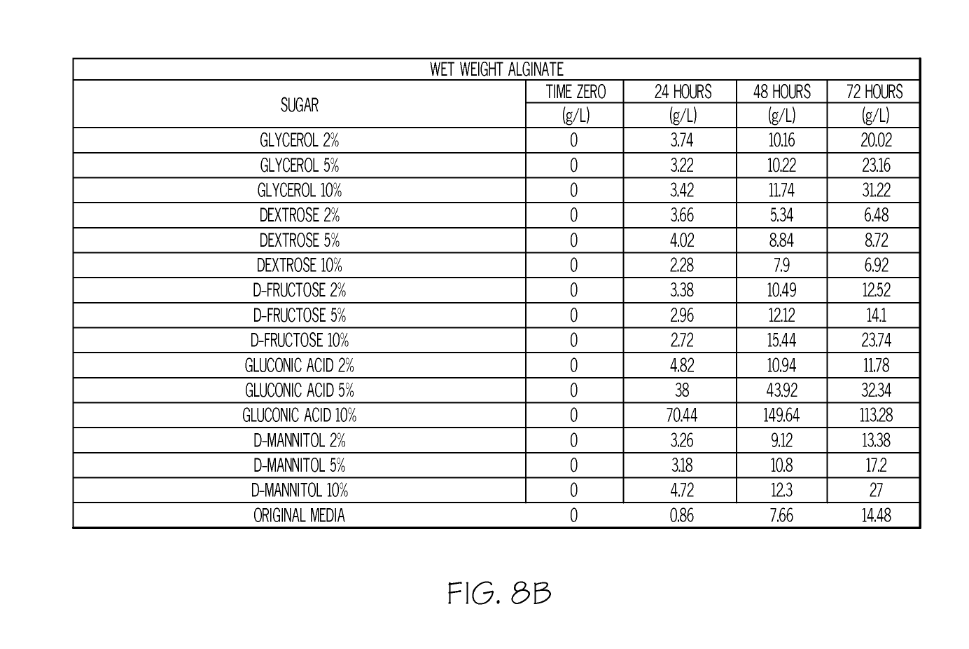

A single colony of VE2 was transferred and grown at 37° C. and 150 rpm overnight in 250 ml PIB media. A stock solution containing 10 g bactopeptone, 10 g pancreatic digest of gelatin, 20 g K₂SO₄, 5 g MgCl₂, and 25 mg triclosan per 50 ml (double concentration solution) was autoclaved along with a separate 50% glycerol solution. Flasks were prepared as shown in FIG. 6. For example, Flask 1 contains 35 ml of the double-concentration solution, 2.8 ml 50% glycerol, and 32.2 ml H₂O. Flasks were then inoculated with 6 ml overnight culture and placed on a 150 rpm shaker at 37° C. and incubated for 72 hours. Samples were taken every 12 hours. A 6 ml sample was removed for OD₆₀₀ and 5 ml was dehydrated with 15 ml 100% ethanol to obtain a wet fiber weight. Fibers were collected carefully and pressed to remove excess ethanol.

Results of the comparison of concentrations of glycerol are shown in FIG. 7. When tested in the range of 2-16% v/v glycerol, it was observed that the concentration of 10% glycerol yielded the highest wet weight of alginate (47 and 42.5 g/liter), as exemplified in Flasks 7 and 8, respectively.

### Example 3

### Effect of Carbon Source on Alginate Production

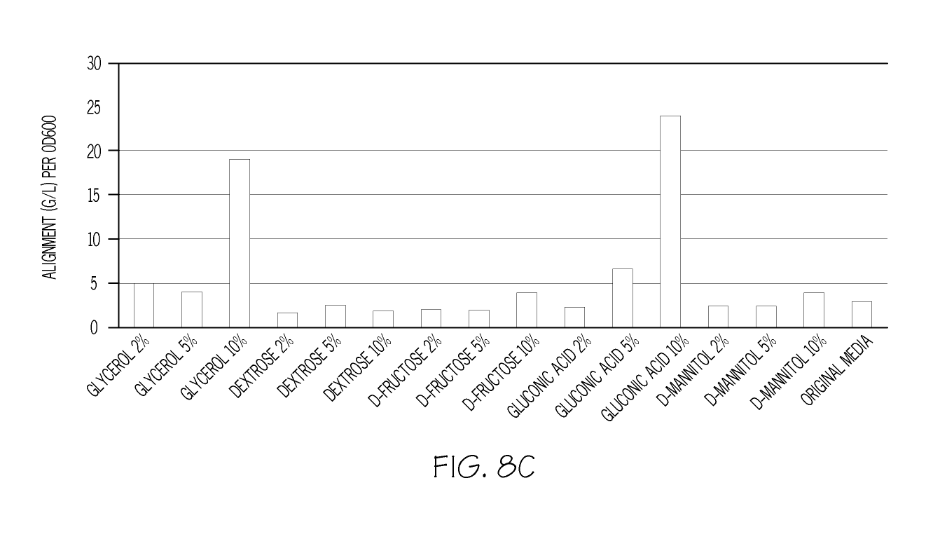

VE2 from frozen stocks was grown on PIA and then an isolated on PIA. A single colony was transferred and grown at 37° C. and 150 rpm overnight in 250 ml PIB media. A stock solution containing 10 g bactopeptone, 10 g pancreatic digest of gelatin, 20 g K₂SO₄, 5 g MgCl₂, and 25 mg triclosan per 500 ml (2× solution) was autoclaved. A 50% glycerol solution was also autoclaved separately. Separate solutions (20 g/100 ml) of gextrose, D-fructose, gluconic acid, and D-mannitol were prepared in autoclaved water. These solutions were then passed through a 0.2 μm filter before use. Flasks were prepared as shown in FIG. 8(A) with exactly 70 ml final total volume. (E.g., Flask 1 contains 35 ml of the 2× solution, 2.8 ml 50% glycerol, and 32.2 ml H₂O, etc.) Flasks were then inoculated with 5 ml overnight culture and placed on a 150 rpm shaker at 37° C., and incubated for 72 hours. Samples were taken every 12 hours. A 6 ml sample was removed for OD₆₀₀ (estimation of cell density) obtained by a spectrophotometer and 5 ml was dehydrated with 15 ml 100% ethanol to obtain a wet fiber weight. Fibers were collected carefully and pressed to remove excess ethanol.

Results are shown in FIGS. 8(B) and 8(C). It was observed that the carbon source that provided the highest wet weight of alginate production was glycerol, at a concentration of about 10%, as exemplified in Flask 3. Flask 10, with 10% gluconate, produced a higher amount of ethanol-precipitatable materials; however a closer examination of the precipitated material shows a difference in texture (more paste-like, with fewer fibers present), indicating likely salt crystal contamination from sodium gluconate.

### Example 4

### Comparison of Media on Alginate Production in a Bioreactor

VE2 was grown at 37° C. overnight in 3 ml PIB From the overnight cultures, a 1 ml sample was transferred to 2 flasks containing 250 ml PIB with 2% glycerol for the first run and 10% glycerol for the second run and incubated at 37° C. at 150 rpm overnight (about 16 hours). A 100 μl sample was also spread onto each of 2 PIA plates and incubated overnight at 37° C. to confirm mucoidy. The bioreactor (Sartorius, Goettingen Germany) was autoclaved to contain 3.5 L PIB or 3 L alginate super media (ASM) for one hour. Comparison of the media contents is shown in Table 1, below:

TABLE 1
Comparison of PIB and ASM media composition, per liter of media

| Components | PIB | ASM |
|---|---|---|
| Bactopeptone (g) | 0 | 10 |
| Pancreatic digest of gelatin (g) | 20 | 10 |
| K₂SO₄ (g) | 10 | 20 |
| MgCl₂ (g) | 1.4 | 5 |
| Triclosan (g) | 0.025 | 0.025 |
| Glycerol (ml) | 20 | 100 |

The pH and dissolved oxygen sensor were carefully calibrated. Bottles of 1M HCl, 1M NaOH, and 0.01% (v/v) Antifoam 204 (Sigma Aldrich) were autoclaved and tubing was connected under sterile conditions. The bioreactor was inoculated using sterile techniques with the flask cultures and sterile media for a final volume of 4 L. This was briefly stirred to fully mix and then a sample was taken for the zero hour time point. The following measurements were recorded: temperature, speed, air, pH both on the bioreactor and independently, acid/base usage, antifoam usage, and alginic acid concentration in both the mannuronic and seaweed alginic acid curves. An OD₆₀₀ of each sample was taken. A 5 ml sample was then used and 25 ml 100% Ethanol was added to estimate production of alginate. This protocol was repeated every four hours using PIB for dilution. The experiment was terminated at 72 hours. Double-concentration PIB media (500 ml) was added at 24 and 48 hours and all changes were recorded.

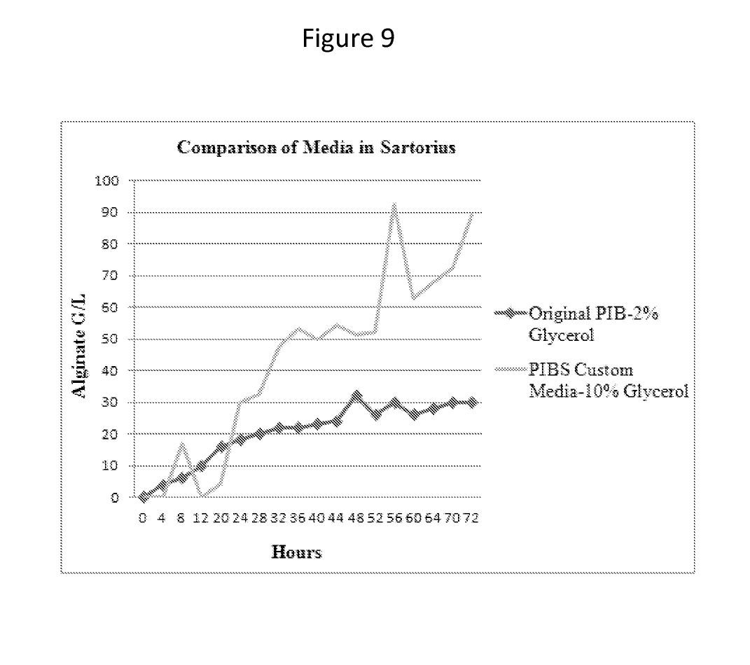

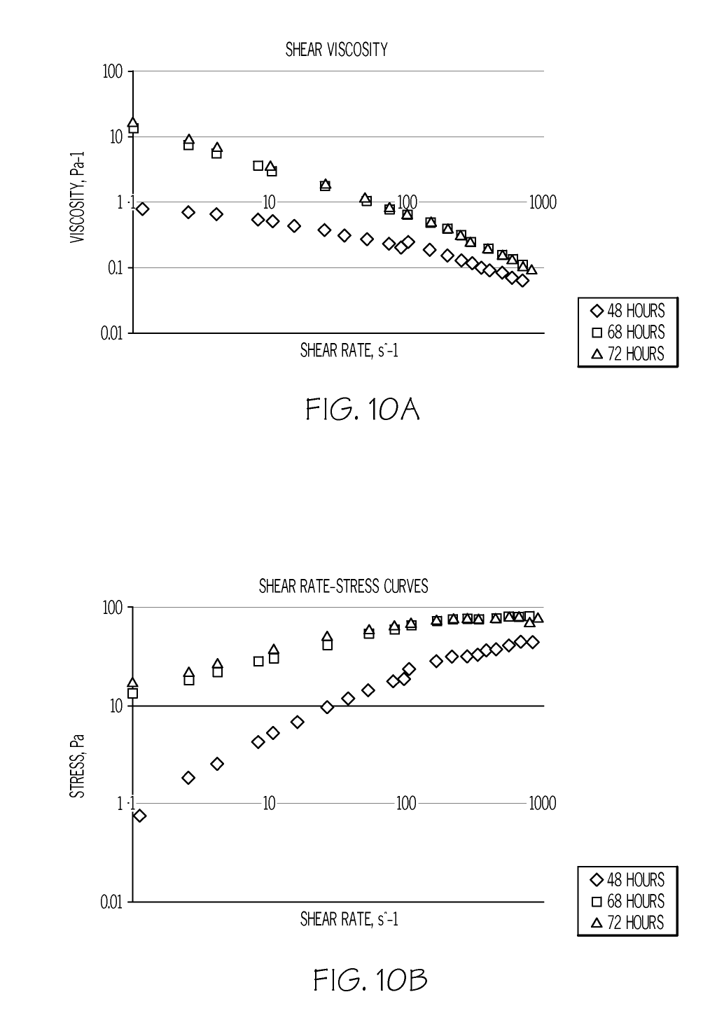

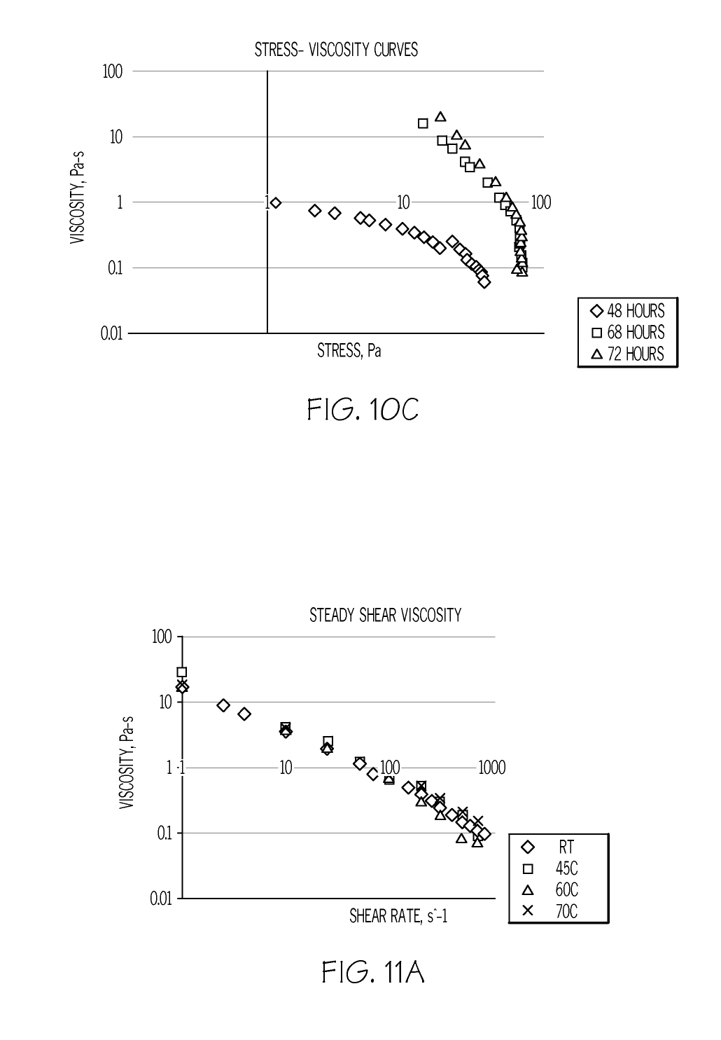

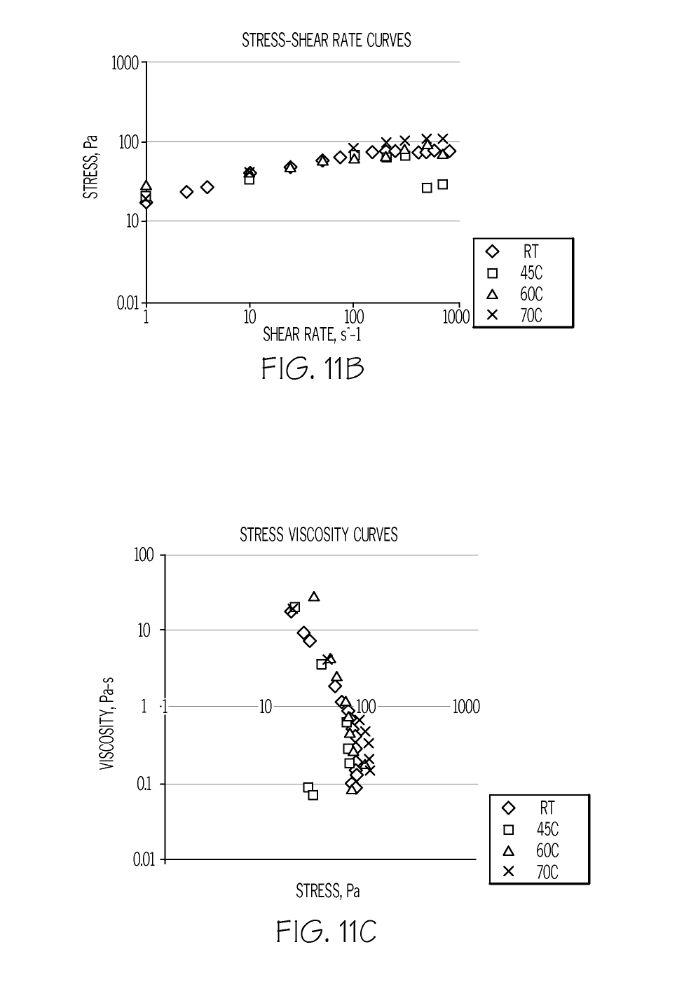

Results from the two bioreactor growth experiments showed that the largest amount of alginate produced using PIB medium was 32 g/L whereas strain VE2 grown with ASM produced a maximum of 92 g/L wet wt. of alginate (See FIG. 9). The weight of the dried alginate fibers was about ⅓ of the wet weight. Since ASM contains 10% glycerol, this gives the conversion rate of 30% from the carbon in glycerol to alginate. Samples of the 48 hour ASM were taken heat-treated at 65° C. for 30, 60, 90, 120 minutes. The 60 minute treatment was optimal to retain rheological properties while preventing alginate degradation. Samples at 68 and 72 hours were treated by heating at 65° C. for 1 hour. All of the treated samples were then sent for rheological studies. See FIGS. 10-11.

Results show that growth time in the bioreactor influences the size of alginate fibers. As time increases, molecular weight of the alginate fibers increases. Surprisingly, the viscosity of the alginate solution in the bioreactor is independent of temperature, which characteristic is distinctive of the alginate produced according to the instant methods.

### Example 5

### Removal of Bacterial Cells Via Ethanol Precipitation of Alginate

Samples were prepared by inoculation of an isolated VE2 colony into 100 ml culture of ASM grown at 37° C. and 150 rpm for 72 hours. Aliquots of 5 ml each were prepared. Triplicate PIA plates were spread with 100 μl of this original culture using a sterile spreader. The remaining samples of this culture were dehydrated with 3 times the volume (15 ml) 100% ethanol. These were solubilized in 5 ml sterile water each on a shaker at 150 rpm and 37° C. overnight. Triplicate PIA plates were spread with 100 μl of these samples (first pass). The remaining samples of this culture were dehydrated with 3 times the volume (15 ml) 100% ethanol. These were solubilized in 5 ml sterile water each on a shaker at 150 rpm and 37° C. overnight. Triplicate PIA plates were spread with 100 μl of these samples (second pass). All plates were incubated at 37° C. for several days to watch for growth. After ethanol dehydration, no colony growth was observed on either the First or Second Pass incubated PIA plates, indicating that alginate precipitated with ethanol is free of bacterial cells.

### Example 6

### Alginate Purification Via Ion Exchange Column

170 g DOWEX™ resin was soaked overnight in 1 L of 10% NaCl solution and then the slurry was poured into a 250 ml column with about 1 inch of glass wool in the bottom. The column was packed and then 1 L each of 10% NaCl, sterile water, and then 0.2 M NaCl was used to condition the column.

A culture of VE2 was grown for 72 hours in ASM media at 37° C. and 150 rpm. The sample was diluted and bacterial cells were removed by centrifugation. The diluted sample was passed through a molecular weight based filter to concentrate the sample, which was then dehydrated in ethanol and rehydrated in water to the original volume of the sample. In the case of the ASM run, it was necessary to dilute with water before proceeding. The resulting supernatant was passed through the DOWEX™ ion exchange column. The addition of the supernatant was facilitated by a peristaltic pump to feed the top and another at the bottom to regulate the output flow rate to about 5 ml/min.

The filtrate was dialyzed again and then collected by ethanol dehydration. Samples were used for HPLC and NMR analysis.

### Example 7

### Alginate Purification Via Ion Exchange Syringe Columns

Columns are prepared as follows: 60 ml syringes without plungers are packed with 25 ml DOWEX™ 1×2-400 resin. Gravity determines the flow rate to be about 4 ml/min.

A culture of VE2 is grown for 72 hours in ASM media at 37° C. and 150 rpm. The sample is processed through Phases I and II as presented herein. The Phase II product is hydrated in water equaling twice the original volume of the sample. The sample is then passed through the prepared columns, dialyzed, and collected by ethanol dehydration. Samples are collected for HPLC and NMR analysis.

The VE2 culture forms several colored bands as the solution runs on the columns. A top layer, brown/tan in color, sits upon the top of the resin and will not travel along the column. Overloading the columns results in a flow through; however, fibers collected from this are significantly whiter than the original fibers. This setup may be used as a quick-clean with smaller volumes of resin.

### Example 8

### HPLC Analysis of Alginate

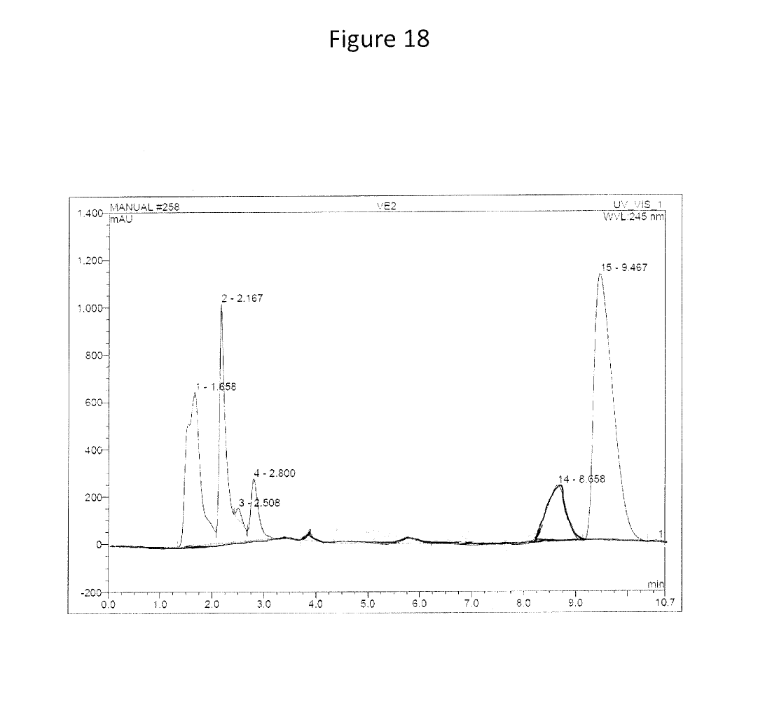

The conventional approach to analyze the alginate content is a carbazole assay that utilizes sulfuric acid to hydrolyze the polysaccharide. The hydrolyzed sugar monomer is then reacted with the carbazole reagent for detection. However, some neutral sugars, such as hexoses and pentoses as well as the acyl groups of uronic acids, can interfere with the specificity of the reaction. Furthermore, even DNA has been shown to affect this assay (Wozniak et al., Alginate is not a significant component of the extracellular polysaccharide matrix of PA14 and PAO1 Pseudomonas aeruginosa biofilms, Prc. Natl. Acad. Sci. USA 100: 7907-12 (2003)). An HPLC protocol was therefore developed by the present inventors for the analysis of bacterial alginate, similar to the protocol for the analysis of seaweed alginate (Wang et al., Analysis of uronic acid compositions in marine brown alga polysaccharides by precolumn derivatization high performance liquid chromatography, Chn. J. Anal. Chem. 37:648-52 (2009)). Seven mg of exopolysaccharides from a bioreactor run processed through Phase II of the instant methods were hydrolyzed in 1 ml 3M trifluoroacetic acid (TFA) for 2 hours at 110° C. TFA was removed from the samples by drying. The hydrolyzed sample was suspended in 300 μl of water and the pH was adjusted to 9 with 0.3 M NaOH. The hydrolyzed alginate was then chromotagged for detection by adding 150 μL 0.5 mol/L 1-phenyl-3-methyl-5-pyrazolone (PMP). The reaction was incubated at 70° C. for 90 min. The pH was then adjusted to 7 with 350 μl 0.3 M HCl. To remove residual PMP, the samples were extracted with 1 ml of chloroform. The PMP-labeled alginate monomers are separated in a phosphate-acetonitrile mobile phase at pH 6.7 and pumped by a Dionex P480 HPLC pump through an Agilent Eclipse XDB-C18 4.6×150 mm column. The PMP-labeled alginate monomers were detected at 245 nm by a Dionex PDA-1000 UV-Vis detector. Chromatograms were generated for known alginate standards (alginic acid from brown algae [61% M/39% G], Sigma-Aldrich A7003) to establish the retention times of PMP-tagged M and G. Under these conditions, PMP-derivatized M and G were detected at 8.8±0.1 min and 9.5±0.1 min respectively (See FIG. 18). The percent of mannuronate (M) to guluronate (M) was calculated based on the relative area of each peak (mAU*min) over the area summation of peaks 14 (G) and 15 (M) of FIG. 18. According to this calculation, the M/G ratio of the VE2 alginate is 70:30.

### Example 9

### NMR Analysis of Alginate

A 100 ml sample was prepared of 0.1% (w/v) alginate (processed according to Phase III, as set forth herein) in water. The sample was adjusted to pH 5.6 with HCl and placed in a 100° C. water bath for 1 hour. (The water bath consisted of a beaker filled with pure autoclaved water on top of a hotplate set to 100° C. and already boiling for a few minutes before the bottle of the sample was added. The bottle cap was vented and the level of the water was adjusted so the level of the alginate solution was just below the level of the water.) The bottle was removed and cooled to touch. The sample was adjusted to pH 3.8 with HCl and placed in a 100° C. water bath for 30 minutes. (Same conditions as above.) The bottle was removed and cooled to touch. The sample was adjusted to a pH between 7 and 8 with NaOH. The sample was then transferred to several 50 ml tubes and stored at −80° C. overnight (on their sides to increase the surface area present). These were then lyophilized and combined. The following samples were prepared:

| | | | | | | | | Total- |
|---|---|---|---|---|---|---|---|---|
| Strains | Fraction | No. | G | M | Ac-3 | Ac-2 | Ac-2,3 | Ac |
| VE2 | 0.8M | 4 | 0.52 | 1.00 | 0.22 | 0.18 | 0.08 | 0.48 |
| | 1M + | 5 | 0.34 | 1.00 | 0.11 | 0.16 | 0.04 | 0.32 |
| | 1.5M | | | | | | | |
| | 2M + | 6 | 0.41 | 1.00 | 0.13 | 0.06 | 0.06 | 0.25 |
| | 2.5M | | | | | | | |

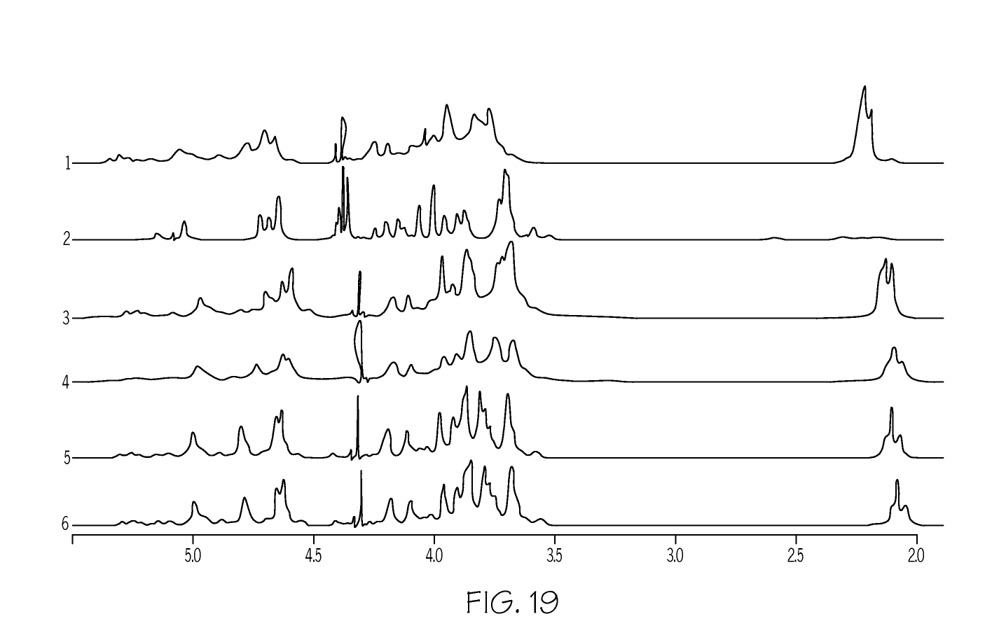

The samples were then sent for NMR analysis. Results are presented in FIG. 19, and show alginate fractions collected through the DOWEX™ column contain G and M monomers of alginate and acetylated residues, suggesting that the DOWEX™ column can be used to enrich alginate by removing contaminating materials.

### Example 10

### Alginate Production by Stable Mucoid Strains of P. aeruginosa

Flasks containing 100 ml ASM are inoculated with a single colony of either P. aeruginosa strain VE2 or strain 581. The flasks are incubated 72 hours at 37° C. and 150 rpm. All 100 ml samples of the liquid cultures are removed for processing. The samples are dehydrated with 95% ethanol to provide first dehydrated alginate fractions. These fractions are then filtered through Dutch weave wire mesh filters (pore size 10 μM) and alginate is collected from the filters. The alginate is then resuspended in 100 ml aliquots of 95% ethanol and filtered again through Dutch weave wire mesh filters to wash the alginate. Washed alginate is collected from the filters.

The alginate samples are then solubilized in 100 ml aliquots of water. Each sample is then dehydrated with 95% ethanol to provide second dehydrated alginate fractions. The second dehydrated alginate fractions are filtered through Dutch weave wire mesh filters and the alginate is collected. Alginate samples are then resuspended in 95% ethanol and filtered again through Dutch weave wire mesh filters to wash the alginate. Washed alginate is collected from the filters.

The washed alginate samples are then solubilized once more in water to provide rehydrated alginate solutions. These solutions are then passed through DOWEX™ ion exchange columns. The columns are washed with NaCl wash solutions. Eluted wash solutions are collected and passed through a molecular sieve filter to concentrate the samples. Samples are then dehydrated with 95% ethanol and filtered through Dutch weave wire mesh filters to collect purified alginate samples.

Results show alginate produced by both strains is comparable in quantity, structure, and purity.

All documents cited are incorporated herein by reference; the citation of any document is not to be construed as an admission that it is prior art with respect to the present invention.

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

What is claimed is:

1. A liquid culture medium for the promotion of alginate production by Pseudomonas aeruginosa (P. aeruginosa) bacterial cells belonging to a strain having a stable mucoid phenotype, the culture medium comprising:
P. aeruginosa bacterial cells belonging to a strain having a stable mucoid phenotype;
about 1% (w/v) enzymatic digest of animal protein;
about 1% (w/v) pancreatic digest of gelatin;
K₂SO₄;
MgCl₂; and
from about 5% (v/v) to about 14% (v/v) glycerol, wherein “about” refers to a variation of ±20% of a value.

2. The liquid culture medium of claim 1, comprising from about 0.5% (w/v) to about 3% (w/v) K₂SO₄.

3. The liquid culture medium of claim 2, comprising from about 1% (w/v) to about 2.5% (w/v) K₂SO₄.

4. The liquid culture medium of claim 1, comprising from about 0.05% (w/v) to about 0.5% (w/v) MgCl₂.

5. The liquid culture medium of claim 4, comprising from about 0.25% (w/v) to about 0.5% (w/v) MgCl₂.

6. The liquid culture medium of claim 1, comprising from about 7% (v/v) to about 12% (v/v) glycerol.

7. The liquid culture medium of claim 1, further comprising about 25 mg/liter triclosan.

8. The liquid culture medium of claim 1, wherein the strain is selected from the group consisting of VE2 and 581.

9. A liquid culture medium for the promotion of alginate production by Pseudomonas aeruginosa (P. aeruginosa) bacterial cells belonging to a strain having a stable mucoid phenotype, the culture medium comprising:
P. aeruginosa bacterial cells belonging to a strain having a stable mucoid phenotype;
about 1% (w/v) enzymatic digest of animal protein;
about 1% (w/v) pancreatic digest of gelatin;
about 2% (w/v) K₂SO₄;
about 0.5% (w/v) MgCl₂;
about 10% (v/v) glycerol; and
about 25 mg/liter triclosan,
wherein “about” refers to a variation of ±20% of a value.

10. A method for producing alginate from Pseudomonas aeruginosa (P. aeruginosa) bacterial cells belonging to a strain having a stable mucoid phenotype, the method comprising:
(a) providing the liquid culture medium of claim 1, wherein the bacterial cells secrete alginate in the liquid culture medium;
(b) dehydrating the liquid culture medium with ethanol to provide a first dehydrated alginate fraction;
(c) filtering the first dehydrated alginate fraction through a Dutch weave wire mesh filter to collect alginate;
(d) resuspending the alginate collected in step (c) in ethanol; and
(e) filtering the alginate resuspended in step (d) through a Dutch weave wire mesh filter to collect washed alginate.

11. The method of claim 10, wherein the Dutch weave wire mesh filter has a pore size of from about 2 μm to about 20 μm.

12. The method of claim 10, further comprising the steps of:
(f) rehydrating the washed alginate collected in step (e) by solubilizing the washed alginate in water to provide a rehydrated alginate solution;
(g) dehydrating the rehydrated alginate solution of step (f) with ethanol to provide a second dehydrated alginate fraction;
(h) filtering the second dehydrated alginate fraction of step (g) through a Dutch weave wire mesh filter to collect alginate;
(i) resuspending the alginate collected in step (h) in ethanol; and
(j) filtering the alginate resuspended in step (i) through a Dutch weave wire mesh filter to collect alginate.

13. The method of claim 12, further comprising the steps of:
(k) drying the alginate collected in step (j); and
(l) milling the alginate dried in step (k).

14. The method of claim 12, wherein the Dutch weave wire mesh filter has a pore size of from about 2 μm to about 20 μm.

15. The method of claim 12, further comprising the steps of:
(k) rehydrating the alginate collected in step (j) by solubilizing the washed alginate in water to provide a rehydrated alginate solution;
(l) passing the rehydrated alginate solution of step (k) through an ion exchange column;
(m) washing the ion exchange column with at least one NaCl wash solution having a concentration of from about 0.2 M to about 3 M and collecting eluted wash solution;
(n) concentrating the eluted wash solution by passing the eluted wash solution through a molecular sieve filter;
(o) dehydrating the eluted wash solution concentrated in step (n) with ethanol to provide a second dehydrated alginate fraction; and
(p) filtering the second dehydrated alginate fraction through a Dutch weave wire mesh filter to collect alginate.

16. The method of claim 15, wherein the Dutch weave wire mesh filter has a pore size of from about 7 μm to about 12 μm.

17. The method of claim 15, further comprising the steps of:
(q) drying the alginate collected in step (p); and
(r) milling the alginate dried in step (q) to provide purified alginate, wherein purity of the purified alginate is greater than about 95%.

18. The method of claim 10, wherein the strain is selected from the group consisting of VE2 and 581 and wherein the purified alginate provided in step (m) is comprised of about 70% mannuronate and about 30% guluronate.

19. The method of claim 10 wherein the ethanol has a concentration of greater than about 85%.

20. The method of claim 10, wherein the liquid culture medium comprises:
P. aeruginosa bacterial cells belonging to a strain having a stable mucoid phenotype;
about 1% (w/v) enzymatic digest of animal protein;
about 1% (w/v) pancreatic digest of gelatin;
about 2% (w/v) K₂SO₄;
about 0.5% (w/v) MgCl₂; and
about 10% (v/v) glycerol.
